(19) United States
(12) Patent Application Publication
(10) Pub. No.: US 2026/0123909 A1
(43) Pub. Date: May 7, 2026

(54) Techniques For Measuring Skin Flap Thickness Using Ultrasound

(71) Applicant: Cochlear Limited, Macquarie University, NSW (AU)

(72) Inventors: Nathan Isaacson, Sydney (AU); Sabina Dabkowski-Chandler, Sydney (AU); Christiane D'hondt, Kruibeke (BE)

(73) Assignee: Cochlear Limited, Macquarie University, NSW (AU)

(21) Appl. No.: 19/118,264

(22) PCT Filed: Oct. 10, 2023
(86) PCT No.: PCT/IB2023/060194

§ 371 Date: Apr. 3, 2025

Related U.S. Application Data
(60) Provisional application No. 63/416,903, filed on Oct. 17, 2022.

(51) Int. Cl. A61B 8/08; A61B 8/00

(52) U.S. Cl. CPC A61B 8/0858; A61B 8/4254

(57) ABSTRACT

A system includes an ultrasound device configured to generate sound waves for use in generating data indicating a measurement of a thickness of a skin flap of a recipient. A method includes determining a location of a measurement of a thickness of a skin flap of the recipient based on a current or expected location of an implantable device underneath the skin flap, and generating the measurement of the thickness of the skin flap using the ultrasound device. A computer-readable storage medium includes instructions stored thereon that cause one or more processors to generate the measurement of the thickness of the skin flap of the recipient based on sonic waves applied to the skin flap, and to generate an indication of whether one or more configurations of an external device are able to be retained on the skin flap based on the measurement.

### CROSS REFERENCE TO RELATED APPLICATION

This patent application claims priority to U.S. provisional patent application 63/416,903, filed Oct. 17, 2022, which is incorporated by reference herein in its entirety.

### FIELD OF THE DISCLOSURE

The present disclosure relates to systems and methods for measuring skin flap thickness of a recipient using ultrasound.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### BRIEF SUMMARY

According to a first aspect of the present invention, a system is provided that comprises an ultrasound device configured to generate sound waves for use in generating data indicating a measurement of a thickness of a skin flap of a recipient.

According to a second aspect of the present invention, a method is provided that comprises determining a location of a measurement of a thickness of a skin flap of a recipient based on a current or expected location of an implantable device underneath the skin flap, and generating the measurement of the thickness of the skin flap using an ultrasound device.

According to a third aspect of the present invention, a non-transitory computer-readable storage medium is provided that comprises instructions stored thereon that, when executed by one or more processors, cause the one or more processors to generate a measurement of a thickness of a skin flap of a recipient based on sonic waves applied to the skin flap, and generate an indication of whether one or more configurations of an external device are able to be retained on the skin flap based on the measurement of the thickness of the skin flap.

### BRIEF DESCRIPTION OF THE DRAWINGS

FIG. 1A depicts a schematic diagram of an exemplary cochlear implant system that can be configured to implement aspects of the techniques presented herein, according to some exemplary embodiments.

FIG. 1B depicts a block diagram of the cochlear implant system of FIG. 1A.

FIG. 2A is a diagram that illustrates a cross-sectional view of an example of an ultrasound device that can be used to generate a measurement of a thickness of a skin flap of an individual at the potential location of an implantable component in the individual.

FIG. 2B is a diagram that illustrates a cross-sectional view of an example of an ultrasound device that can be used to generate a measurement of a thickness of a skin flap of an individual at the location of an implantable component in the individual.

FIG. 2C is a diagram that illustrates a cross-sectional view of the ultrasound device of FIG. 2B and a tool that is used to align the ultrasound device with the implantable component.

FIG. 3A is a flow chart that illustrates examples of operations that can be performed to measure skin flap thickness using ultrasound, according to another embodiment.

FIG. 3B is a flow chart that illustrates examples of operations that can be performed to determine the suitability of an external component of an implant system for a recipient, according to another embodiment.

FIG. 4 illustrates an example of a suitable computing system with which one or more of the disclosed embodiments can be implemented.

### DETAILED DESCRIPTION

Merely for ease of description, the techniques presented herein are primarily described herein with reference to an illustrative medical device, namely a cochlear implant system. However, it is to be appreciated that the techniques presented herein may also be used with a variety of other medical devices that, while providing a wide range of therapeutic benefits to recipients, patients, or other users, may benefit from the teachings herein used in other medical devices. For example, any techniques presented herein described for one type of hearing prosthesis, such as a cochlear implant system, corresponds to a disclosure of another embodiment of using such teaching with another hearing prostheses, including bone conduction devices (percutaneous, active transcutaneous and/or passive transcutaneous), middle ear auditory prostheses, direct acoustic stimulators, and also utilizing such with other electrically simulating auditory prostheses (e.g., auditory brain stimulators), etc. The techniques presented herein may also be used with vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation, etc.

While the teachings detailed herein will be described for the most part with respect to hearing prostheses, in keeping with the above, it is noted that any disclosure herein with respect to a hearing prosthesis corresponds to a disclosure of another embodiment of utilizing the associated teachings with respect to any of the other prostheses noted herein, whether a species of a hearing prosthesis, or a species of a sensory prosthesis, such as a retinal prosthesis. In this regard, any disclosure herein with respect to evoking a hearing percept corresponds to a disclosure of evoking other types of neural percepts in other embodiments, such as a visual/sight percept, a tactile percept, a smell precept or a taste percept, unless otherwise indicated and/or unless the art does not enable such. Any disclosure herein of a device, system and/or method that is used to or results in ultimate stimulation of the auditory nerve corresponds to a disclosure of an analogous stimulation of the optic nerve utilizing analogous components, methods, and systems.

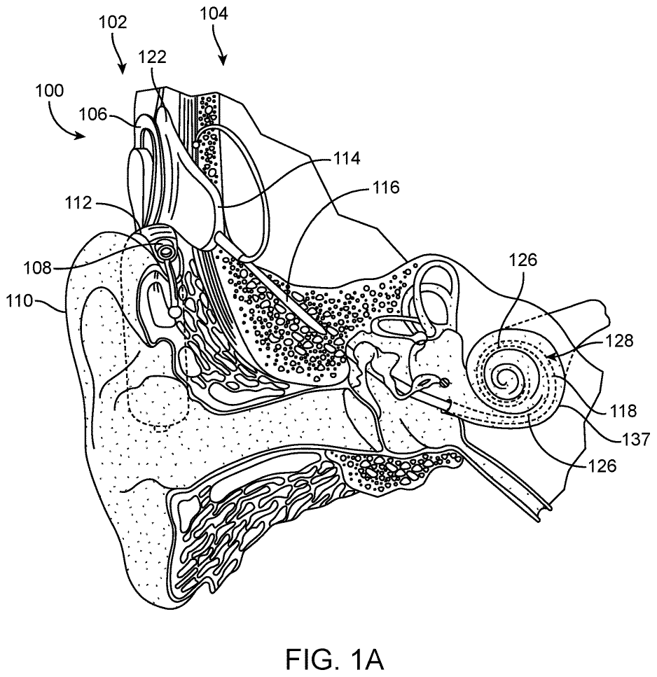

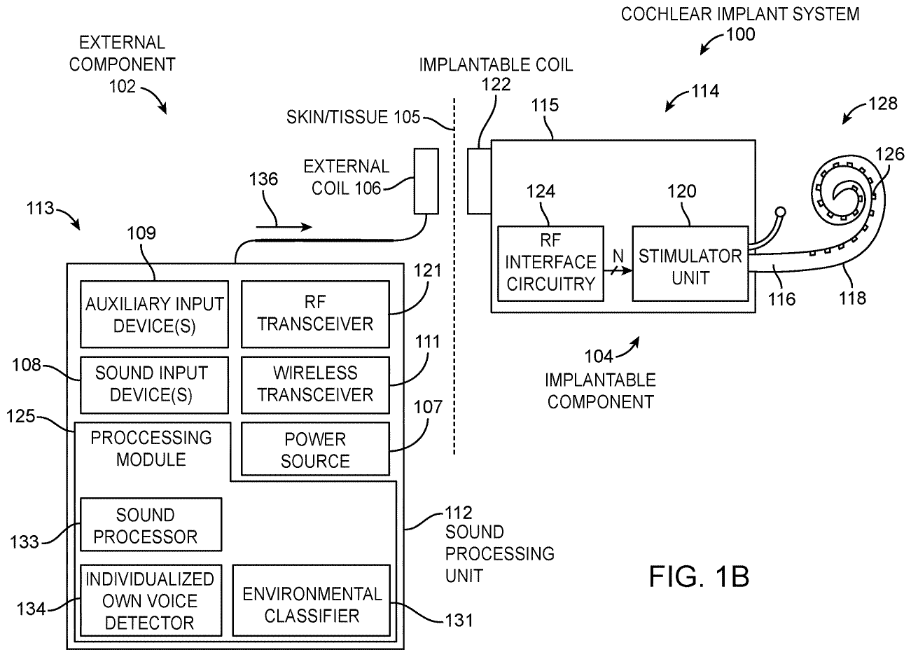

FIG. 1A is a schematic diagram of an exemplary cochlear implant system 100 configured to implement aspects of the techniques presented herein. FIG. 1B is a block diagram of the cochlear implant system 100 of FIG. 1A. For ease of illustration, FIGS. 1A and 1B will be described together. The cochlear implant system 100 comprises an external component 102 and an internal/implantable component 104. The external component 102 is directly or indirectly attached to the body of the recipient and typically comprises an external coil 106 and, generally, a magnet (not shown in FIGS. 1A-1B) fixed relative to the external coil 106. The external component 102 also comprises one or more input elements/devices 113 for receiving input signals at a sound processing unit 112. In this example, the one or more input devices 113 include sound input devices 108 (e.g., microphones positioned by auricle 110 of the recipient, telecoils, etc.) configured to capture/receive input signals, one or more auxiliary input devices 109 (e.g., audio ports, such as a Direct Audio Input (DAI), data ports, such as a Universal Serial Bus (USB) port, cable port, etc.), and a wireless transmitter/receiver (transceiver) 111, each located in, on, or near the sound processing unit 112.

The sound processing unit 112 also includes, for example, at least one power source 107, a radio-frequency (RF) transceiver 121, and a processing module 125. The processing module 125 comprises a number of elements, including an environmental classifier 131, a sound processor 133, and an individualized own voice detector 134. Each of the environmental classifier 131, the sound processor 133, and the individualized own voice detector 134 may be formed by one or more processors (e.g., one or more Digital Signal Processors (DSPs), one or more processing cores, etc.), firmware, software, etc. arranged to perform operations described herein. That is, the environmental classifier 131, the sound processor 133, and the individualized own voice detector 134 may each be implemented as firmware elements, partially or fully implemented with digital logic gates in one or more application-specific integrated circuits (ASICs), partially or fully in software, etc.

In the examples of FIGS. 1A and 1B, the sound processing unit 112 is a behind-the-ear (BTE) sound processing unit configured to be attached to, and worn adjacent to, the recipient's ear. However, it is to be appreciated that sound processing unit 112 may have other arrangements, such as an off the ear (OTE) processing unit (e.g., a component having a generally cylindrical shape and which is configured to be magnetically coupled to the recipient's head), etc., a mini or micro-BTE unit, an in-the-canal unit that is configured to be located in the recipient's ear canal, a body-worn sound processing unit, etc.

In the exemplary embodiment of FIGS. 1A and 1B, the implantable component 104 comprises an implant body (main module) 114, a lead region 116, and an intra-cochlear stimulating assembly 118, all configured to be implanted under the skin/tissue (tissue) 105 of the recipient. The implant body 114 generally comprises a hermetically-sealed housing 115 in which RF interface circuitry 124 and a stimulator unit 120 are disposed. The implant body 114 also includes an internal/implantable coil 122 that is generally external to the housing 115, but which is connected to the RF interface circuitry 124 via a hermetic feedthrough (not shown in FIG. 1B).

As noted, stimulating assembly 118 is configured to be at least partially implanted in the recipient's cochlea 137. Stimulating assembly 118 includes a plurality of longitudinally spaced intra-cochlear electrical stimulating contacts (electrodes) 126 that collectively form a contact or electrode array 128 for delivery of electrical stimulation (current) to the recipient's cochlea. Stimulating assembly 118 extends through an opening in the recipient's cochlea (e.g., cochleostomy, the round window, etc.) and has a proximal end connected to stimulator unit 120 via lead region 116 and a hermetic feedthrough (not shown in FIG. 1B). Lead region 116 includes a plurality of conductors (wires) that electrically couple the electrodes 126 to the stimulator unit 120.

As noted, the cochlear implant system 100 includes the external coil 106 and the implantable coil 122. The coils 106 and 122 are typically wire antenna coils each comprised of multiple turns of electrically insulated single-strand or multi-strand wire. Generally, a magnet is fixed in position relative to each of the external coil 106 and the implantable coil 122, but the magnet may rotate or change orientation. In some embodiments, the external component 102 and/or the implantable component 104 can include magnet assemblies that each have more than one magnet component. The magnets fixed relative to the external coil 106 and the implantable coil 122 facilitate the operational alignment of the external coil with the implantable coil. This operational alignment of the coils 106 and 122 enables the external component 102 to transmit data, as well as possibly power, to the implantable component 104 via a closely-coupled wireless link formed between the external coil 106 with the implantable coil 122. In certain examples, the closely-coupled wireless link is a radio frequency (RF) link. However, various other types of energy transfer, such as infrared (IR), electromagnetic, capacitive and inductive transfer, may be used to transfer the power and/or data from an external component to an implantable component and, as such, FIG. 1B illustrates only one exemplary arrangement.

As noted above, sound processing unit 112 includes the processing module 125. The processing module 125 is configured to convert input audio signals into stimulation control signals 136 for use in stimulating a first ear of a recipient (i.e., the processing module 125 is configured to perform sound processing on input audio signals received at the sound processing unit 112). Stated differently, the sound processor 133 (e.g., one or more processing elements implementing firmware, software, etc.) is configured to convert the captured input audio signals into stimulation control signals 136 that represent electrical stimulation for delivery to the recipient. The input audio signals that are processed and converted into stimulation control signals may be audio signals received via the sound input devices 108, signals received via the auxiliary input devices 109, and/or signals received via the wireless transceiver 111.

In the embodiment of FIG. 1B, the stimulation control signals 136 are provided to the RF transceiver 121, which transcutaneously transfers the stimulation control signals 136 (e.g., in an encoded manner) to the implantable component 104 via external coil 106 and implantable coil 122. That is, the stimulation control signals 136 are received at the RF interface circuitry 124 via implantable coil 122 and provided to the stimulator unit 120. The stimulator unit 120 is configured to utilize the stimulation control signals 136 to generate electrical stimulation signals (e.g., current signals) for delivery to the recipient's cochlea via one or more stimulating contacts 126. In this way, cochlear implant system 100 electrically stimulates the recipient's auditory nerve cells, bypassing absent or defective hair cells that normally transduce acoustic vibrations into neural activity, in a manner that causes the recipient to perceive one or more components of the input audio signals.

Skin flap thickness typically refers to the thickness of a portion of the external soft tissue of an individual that overlies a rigid underlying structure, such as a bone or an implanted device. In the context of FIGS. 1A-1B, the thickness of the layer of the soft tissue/skin 105 (i.e., the skin flap thickness) that lies between the implantable coil 122 and the external coil 106 is a significant factor that influences the retention of the external coil 106 to the head of the recipient and the RF link performance between the external and implantable coils. For cochlear implant systems that have off-the-ear (OTE) sound processors, the skin flap thickness between the implantable coil and the external coil is often the main factor that influences the retention of the external component of the cochlear implant system to the head of the recipient.

Individuals who are considering receiving cochlear implant systems often want to know in advance of cochlear implant surgery if they are candidates for an OTE sound processor. A recipient of a cochlear implant system may be disappointed if a determination is made after implantation of the implantable component of the cochlear implant system that available OTE sound processors do not provide adequate retention for the recipient's skin flap thickness. For this reason, it is often desirable to determine the skin flap thickness of an individual at a location (e.g., on the individual's head) over an implantable coil of a cochlear implant system or at a proposed location to implant an implantable coil of a cochlear implant system.

The thickness of the skin flap of an individual at a location or at a potential location of an implantable component of an implant system can be used for several purposes. As an example, the external components of some types of cochlear implant systems can only be retained on an individual's head by a magnet in an implantable component if the individual has a skin flap thickness over the implantable component that is within a supported range of skin flap thicknesses. According to some embodiments disclosed herein, the thickness of the skin flap of an individual can be measured using an ultrasound device. The ultrasound device can be used to measure the skin flap thickness of an individual over a component implanted in the individual (e.g., the implanted coil of a cochlear implant system) or over the potential location of an implantable component (e.g., a predicted implantable coil site for a candidate of a cochlear implant system). Ultrasound is an imaging technique that typically uses sound waves (e.g., high frequency sound waves) to produce images of structures. An ultrasound device can include an ultrasound transducer that generates the sound waves. The sound waves generated by an ultrasound device can indicate the difference in density between the soft tissue of an individual and a rigid surface (such as metal or bone) underlying the soft tissue. The difference in density can be used to calculate the distance from the surface of the ultrasound device to the rigid surface underlying the soft tissue. This distance can be used as a measurement of skin flap thickness. The measurement may be, for example, an estimate of skin flap thickness. The ultrasound device can measure the skin flap thickness of an individual prior to implantation of an implantable component of an implant system (e.g., a cochlear implant system) in the individual and/or after the implantable component of the implant system has been implanted in the individual. As used herein, the term “recipient” includes individuals that have received an implantable component of an implant system (e.g., have had an implantable component surgically implanted) and individuals that are candidates for receiving an implantable component of an implant system in the future (e.g., are being evaluated for receiving an implantable component).

The measurement of the skin flap thickness at a location on an individual can be used to predict whether the external component of an implant system can be retained by the implantable component of the implant system after implantation at the measured location. The measurement of the thickness of the skin flap of an individual can be used to determine whether a particular combination or configuration of external and implantable components of an implant system (such as a cochlear implant system) can be retained on the individual through the skin flap. The measurement of the skin flap thickness of an individual can also be used to predict the magnetic field strength required for adequate retention of an external component of an implant system by the implantable component of the implant system.

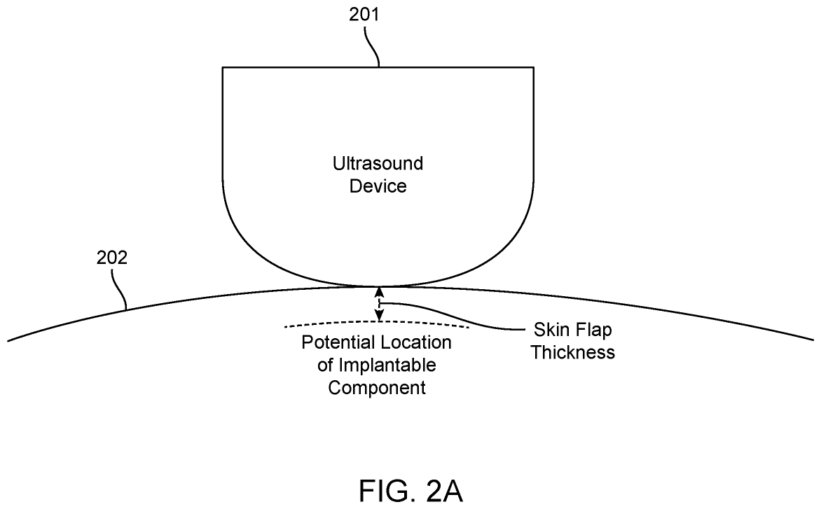

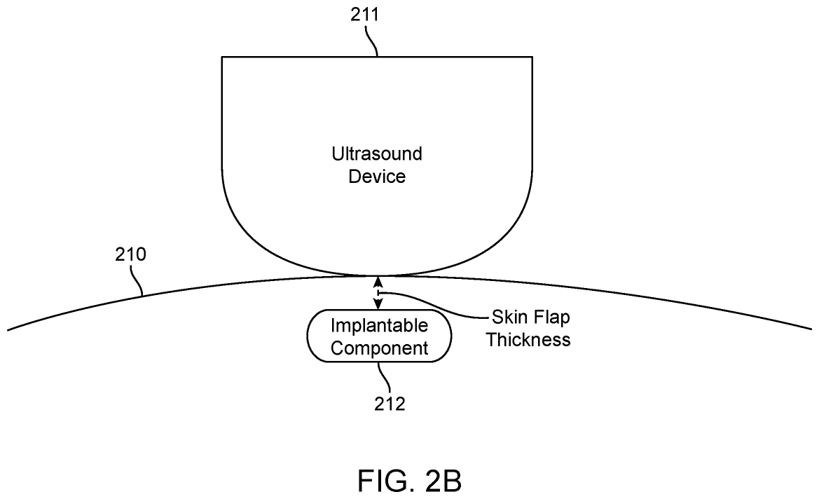

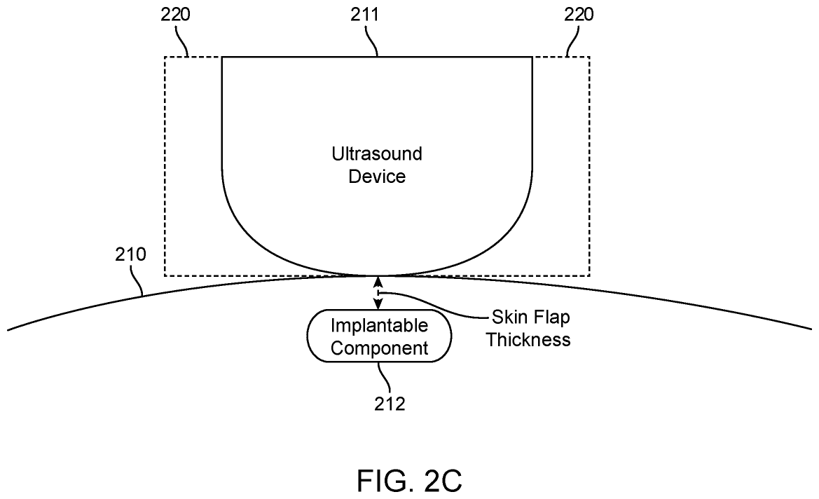

FIG. 2A is a diagram that illustrates a cross-sectional view of an example of an ultrasound device that can be used to generate a measurement of a thickness of a skin flap of an individual at the potential location of an implantable component in the individual. FIG. 2A illustrates a handheld ultrasound device 201 that generates sonic waves for generating a measurement of a skin flap thickness of an individual. Ultrasound device 201 can be used to generate a measurement of a skin flap thickness on the head 202 of the individual at the potential location of an implantable component in the individual, as shown in FIG. 2A. A measurement of the skin flap thickness of the individual at the potential location of a cochlear implant system can be used by a clinician to determine whether an external component (e.g., an off the ear (OTE) sound processor) can be retained on the individual at the potential location of the implantable component of the cochlear implant system. The ultrasound device 201 can be used to perform a measurement of skin flap thickness before surgery to implant an implantable component in the individual. Providing a clinician with a measurement of the skin flap thickness prior to implantation of an implantable component (such as an implantable component of a cochlear implant system) allows the clinician to be able to successfully manage a recipient's expectations and to achieve an improved overall experience. The measurement of the skin flap thickness can also be used by a surgeon to determine which implant system to implant in an individual (e.g., an OTE sound processor, if preferred by an individual). The measurement of the skin flap thickness can also be used by a surgeon to determine whether a skin flap thinning procedure would be beneficial for the individual prior to implantation of an implant system.

FIG. 2B is a diagram that illustrates a cross-sectional view of an example of an ultrasound device that can be used to generate a measurement of a thickness of a skin flap of an individual at the location of an implantable component in the individual. FIG. 2B illustrates an ultrasound device 211 (or a portion thereof such as an ultrasonic transducer) that generates sonic waves for use in generating a measurement of a skin flap thickness in a head 210 of an individual. Ultrasound device 211 can be used to generate a measurement of a skin flap thickness (shown in FIG. 2B) on the head 210 of the individual above the location of an implantable component 212 of an implant system (such as a cochlear implant system) that has been implanted underneath the skin flap of the individual.

FIG. 2C is a diagram that illustrates a cross-sectional view of the ultrasound device of FIG. 2B and a tool that is used to align the ultrasound device with the implantable component. In the example of FIG. 2C, the ultrasound device 211 is placed in a tool 220 that is used to align the ultrasound device 211 with the implantable component 212. As an example, the tool 220 can include a magnetic device that is used to align the ultrasound device 211 with a magnet in the implantable component 212. The tool 220 can, for example, be removed prior to taking the ultrasound measurement of the skin flap thickness using ultrasound device 211. The ultrasound device 211 can be part of a system that includes the tool 220 having the magnetic device used to align the ultrasound device 211 (e.g., the ultrasonic transducer) with a magnet in the implantable component 212. The tool 220 can, for example, be placed around an external component of an implant system (e.g., around external coil 106 of FIGS. 1A-1B), and then the external component of the implant system can be removed and replaced with the ultrasound device 211 and/or the ultrasonic transducer of ultrasound device 211. The ultrasound device 211 can then perform the measurement of the skin flap thickness as disclosed herein.

The tool 220 of FIG. 2C can have a variety of shapes. For example, the tool 220 can have a cylindrical or box shape. The tool can include, for example, a ring made of plastic or other materials that fits around the external component of an implant system, such as external coil 106 or an OTE sound processor. The magnetic device in the ultrasound device 211 or tool 220 can have any shape. For example, the magnetic device can have a shape similar to a sound processor magnet. As another example, the magnetic device in the ultrasound device 211 or tool 220 can have a toroidal shape that facilitates alignment with a circular shaped magnet implanted in a recipient.

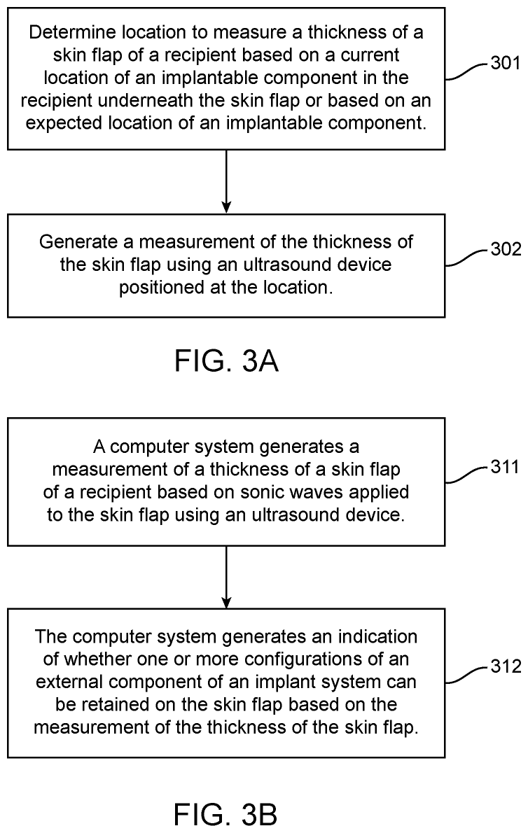

FIG. 3A is a flow chart that illustrates examples of operations that can be performed to measure skin flap thickness using ultrasound, according to an embodiment. In operation 301, a location is determined to measure a thickness of a skin flap of a recipient based on a current location of an implantable component in the recipient underneath the skin flap or based on an expected location of an implantable component. In operation 302, a measurement of the thickness of the skin flap is generated using an ultrasound device positioned at the location. The location may, for example, be on the head of a recipient, or a potential recipient, of an implant system, such as a cochlear implant system. The ultrasound device can be used to conduct a consistent measurement of the skin flap thickness at the location using the same measurement related parameters, for example, the angle of the ultrasound device relative to the skin, the pressure applied to the skin by the ultrasound device, etc.

The ultrasound device can, for example, have an ultrasonic transducer that can be used to conduct an ultrasound scan of the skin flap and that can be used to collect data from the ultrasound scan. For example, the ultrasonic transducer can generate sonic waves for application to the skin flap, and the ultrasonic transducer can then collect sonic waves that are received (e.g., reflected) from the skin flap in response to the sonic waves applied to the skin flap. The ultrasound device can then generate data that is indicative of a thickness of the skin flap based on the sonic waves that are received from the skin flap.

The ultrasound device can include a computing system. Alternatively, the ultrasound device can be in communication with a separate computing system that has a wired or wireless communication interface with the ultrasound device. As examples, the ultrasound device, or the separate computing system, can include a wireless handheld device, a desktop computer, a tablet computer, a laptop computer, and/or any other type of computing system. The ultrasound device, or the separate computing system, can include one or more processor circuits, such as one or more microprocessor integrated circuits, that are configured to run software. The software can be configured to receive and interpret data from the ultrasound device/transducer and to perform calculations for determining a measurement of a thickness of a skin flap of a recipient based on the received data indicating sonic waves generated and/or received by the ultrasound device.

The software can present data related to the measurement of the thickness of the skin flap to a user in one or more user interfaces. The software can, for example, be run on a handheld device, or another type of computing system, having a display screen that presents the one or more user interfaces. The data related to the measurement of the skin flap thickness can, for example, be displayed to a user by the ultrasound device directly or on a separate computing system (e.g., on a display screen). The user interface can display the data to the user in an easy-to-understand format, so that a clinician can use the ultrasound device without having to undergo specialized ultrasound technician training. In some embodiments, the ultrasound device and/or the separate computing system can display an ultrasound image of the skin flap. In other embodiments, the ultrasound device and/or the separate computing system do not show an ultrasound image, so that a user does not need to have specialized knowledge of sonography to interpret the data presented to the user.

FIG. 3B is a flow chart that illustrates examples of operations that can be performed to determine the suitability of an external component of an implant system for a recipient, according to another embodiment. In operation 311, a computer system generates a measurement of a thickness of a skin flap of a recipient based on sonic waves applied to the skin flap using an ultrasound device. In operation 312, the computer system generates an indication of whether one or more configurations of an external component of an implant system can be retained on the skin flap based on the measurement of the thickness of the skin flap. As examples, the computer system can generate the indication based on the measurement of the thickness of the skin flap and/or one or more of magnetic field strengths of the external component and/or the implantable component of the implant system, a recommended skin flap thickness range for the combination of the external component and the implantable component of the implant system, the weight of the external component, the size of the external component, etc.

The ultrasound device and/or the separate computing system can perform internal image processing and calculations to generate a measurement of the thickness of the skin flap based on the sonic waves generated and/or received by the ultrasonic transducer. The ultrasound device and/or the separate computing system can then provide data to a user based on the measurement of the thickness of the skin flap. The data related to the measurement of the thickness of the skin flap that is provided to the user can, for example, include the measurement (e.g., an estimate) of the skin flap thickness, a recommendation for an external component associated with an implant system (such as a cochlear implant system) for the recipient, a recommendation for a magnetic configuration of an external component of an implant system that can be retained on the skin flap by an implantable component of the implant system, or eligibility of the recipient for a particular external component (such as an OTE sound processor) that is associated with an implant system. The magnetic configuration can include magnets polarized in different directions or with a different number of magnetic poles, and/or a variation in a single magnetic field strength value.

The ultrasound device and/or the separate computing system can also calculate whether a measured skin flap thickness is supported by a range of magnet configurations that are available in external and implantable components of an implant system (such as a cochlear implant system). The measurement of the skin flap thickness of a recipient generated using an ultrasound device can be used to determine which magnetic configurations of external and implantable components of an implant system, such as a cochlear implant system, are recommended for the recipient having the measured skin flap thickness. The measurement of the skin flap thickness of a recipient using an ultrasound device can also be used, for example, to determine a suitable magnetic field strength for the skin flap and/or to assist a clinician in selecting a combination of external and implantable components of an implant system having the suitable magnetic field strength. The ultrasound device and/or the separate computing system can provide data to the user (e.g., on a display screen) that indicates one or more recommended magnet configurations for an implantable component and/or that indicates recommended external and implantable components of an implant system based on the measured skin flap thickness and/or the suitable magnetic field strength. The ultrasound device and/or the separate computing system can determine a suitability of one or more combinations of external and implantable components of an implant system for a recipient at a current or expected location of the implantable component based on pre-determined criteria. The pre-determined criteria can include, for example, whether the measured skin flap thickness falls within a defined skin flap thickness range for which the one or more combinations are suitable and/or the expected daily activities of the recipient (e.g., sporting activities, sedentary activities, etc.).

The measurement of the skin flap thickness of a recipient can, for example, be used to determine if the recipient is eligible for a particular external component (e.g., an OTE sound processor) of a cochlear implant system or for a particular implantable or external component of another type of implant system. For example, if a recipient has a large skin flap thickness, the ultrasound device and/or the separate computing system can provide information indicating that the recipient would be unlikely to achieve sufficient retention with a particular external component (e.g., an OTE sound processor). The ultrasound device and/or the separate computing system can also, or alternatively, provide a recommendation for an external component of an implant system that would likely be retained on the recipient based on the measurement of the skin flap thickness. This information and/or recommendation can be provided to the user, for example, on a display screen. A clinician can use this information and/or recommendation to manage expectations prior to implantation of an implantable component or prior to upgrading the external component of an implant system.

As another example, the ultrasound device and/or the separate computing system can provide a more targeted recommendation, such as a recommendation for a surgeon to perform skin flap thinning on a particular recipient based on a measurement of the skin flap thickness using ultrasound. This recommendation can be provided for a recipient having a large skin flap thickness. If a measurement from the ultrasound device indicates that a recipient has a large skin flap thickness pre-operatively, and the recipient is made aware and still desires a particular external component that requires a smaller skin flap thickness, a surgeon may choose to perform skin flap thinning surgically on the recipient or may use a different external or implantable component of an implant system.

As still another example, a measurement of skin flap thickness using ultrasound can be used to provide guidance on a surgical approach for implantation of an implantable component in a recipient. As a specific example, a skin flap thickness measurement can be used to determine where to place the implantable coil of a cochlear implant system (e.g., under the periosteum, on top of the periosteum, or on top of a muscle) and to assist a clinician in selecting a suitable magnetic field strength for a particular recipient and sound processor combination.

In some embodiments, an ultrasound device that is used to measure skin flap thickness can include a magnetic device (e.g., a magnet). The magnetic device in the ultrasound device can, for example, be used to consistently align the ultrasonic transducer with a magnet implanted in a recipient, such as a magnet fixed to implantable coil 122 of FIGS. 1A-1B. The magnetic device can be, for example, a magnet built into the ultrasound device.

According to another embodiment, the ultrasound device can perform ultrasonic imaging of a recipient to generate an ultrasound image, and then the ultrasound image can be analyzed to determine a location of one or more landmarks on an implantable component under the skin flap relative to the ultrasound device. For example, the ultrasound image can be analyzed to locate the center point of a component implanted in the recipient (such as implantable coil 122, an implanted magnet, or a radio-frequency coil) to guide a user in placing the ultrasound device on the recipient over the implanted component. The ultrasound device or a separate computing system can, for example, display ultrasonic images of the recipient using sonic waves sensed by the ultrasound device to guide the user in placing the ultrasound device on the recipient. The ultrasonic images can, for example, be similar to a two-dimensional heat map. The ultrasonic images can display the location of an implanted magnet, an implanted coil, and/or any other component implanted in a recipient.

The ultrasound device can, for example, have a radio-frequency (RF) coil that uses transmitted and/or received power as a measurement for aligning the ultrasound device with an implanted RF coil, such as implantable coil 122. In other examples, the ultrasound device can use one, two, or more of magnetic, ultrasonic imaging, and/or RF coil techniques to locate a landmark of a component implanted in a recipient (e.g., the center of the RF coil) or to locate an anatomical landmark of the recipient. According to still another embodiment, an ultrasound device and/or an ultrasonic transducer that is part of an ultrasound device can be incorporated into an external component of an implant system, such as cochlear implant system.

According to other embodiments, an ultrasound device that is used to generate measurements of skin flap thickness can include, or be in communication with, a pressure sensor. The pressure sensor can measure the pressure that the ultrasound device applies to the skin flap of the recipient that is measured by the ultrasound device. The pressure sensor can be used to indicate when the ultrasound device is applying a predetermined pressure, a minimum pressure, or a pressure within a predetermined range, to the skin flap. The predetermined pressure, or a range of pressures, can be selected to cause measurements of the skin flap thickness using the ultrasound device at the predetermined pressure, or within the ranges of pressures, on the skin flap to be accurate and consistent.

According to still other embodiments, the ultrasound device (or another connected intermediary device) can communicate with a computing system that runs clinical software that automatically records skin flap thickness measurements made using the ultrasound device. Alternatively, measurements of skin flap thickness can be input manually by a user into a computing system running clinical software. The clinical software running on the computing system can perform any desired functions with measurements of the skin flap thicknesses of one or more recipients made using the ultrasound device. As examples, the clinical software can track changes in the skin flap thickness of a recipient (e.g., a recipient of a cochlear implant system) over time, and/or prompt a clinician when a change in an implanted and/or external magnet is recommended. The clinical software can, for example, be used to make magnetic field strength recommendations for implant systems, such as cochlear implant system 100 of FIGS. 1A-1B, and external components, such as sound processors.

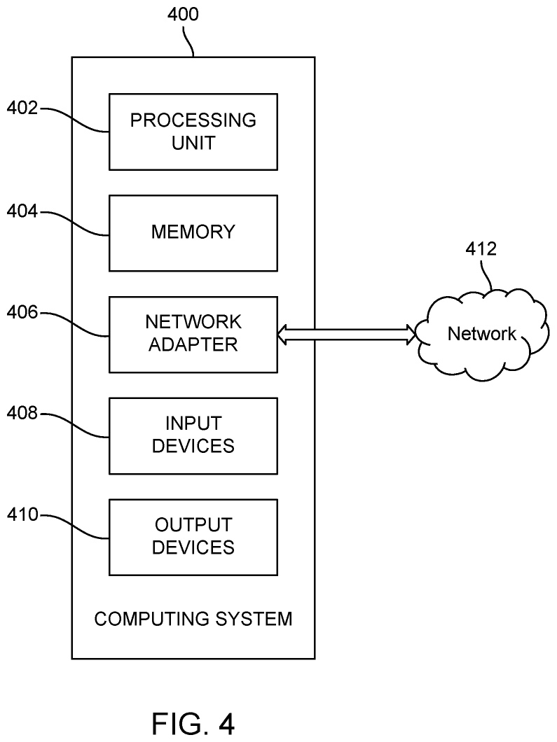

FIG. 4 illustrates an example of a suitable computing system 400 that can perform any of the operations or functions disclosed herein. For example, computing system 400 can be used to generate measurements of a thickness of a skin flap of a recipient based on sonic waves applied to the skin flap by an ultrasound device, as disclosed herein. In addition, computing system 400 can generate an indication of whether one or more configurations of an external device of an implant can be retained on the skin flap based on a measurement of the thickness of the skin flap using ultrasound, as disclosed herein. Computing system 400 is an example of any of the computing systems disclosed herein, such as a computing system in an ultrasound device or a computing system separate from the ultrasound device. Computing systems, environments, or configurations that can be suitable for use with examples disclosed herein include, but are not limited to, personal computers, server computers, hand-held devices, laptop devices, multiprocessor systems, microprocessor-based systems, programmable consumer electronics (e.g., smart phones), network computers, minicomputers, mainframe computers, tablets, distributed computing environments that include any of the above systems or devices, and the like. The computing system 400 can be a single virtual or physical device operating in a networked environment over communication links to one or more remote devices. The remote device can be an auditory prosthesis (e.g., the auditory prosthesis of FIGS. 1A-1B), an ultrasound device, a pressure sensor, a personal computer, a server, a router, a network personal computer, a peer device or other common network node.

Computing system 400 includes at least one processing unit 402 and memory 404. The processing unit 402 includes one or more hardware or software processors (e.g., Central Processing Units) that can obtain and execute instructions. The processing unit 402 can communicate with and control the performance of other components of the computing system 400. The memory 404 is one or more software-based or hardware-based computer-readable storage media operable to store information accessible by the processing unit 402.

The memory 404 can store instructions executable by the processing unit 402 to implement applications (software) or cause performance of any of the functions or operations disclosed herein, as well as store other data. The memory 404 can be volatile memory (e.g., random access memory or RAM), non-volatile memory (e.g., read-only memory or ROM), or combinations thereof. The memory 404 can also include one or more removable or non-removable storage devices. The memory 404 can include transitory memory and/or non-transitory computer-readable storage media. Non-transitory computer-readable storage media is tangible computer-readable storage media that stores data for access at a later time, as opposed to media that only transmits propagating electrical signals, such as wires. In examples, the memory 404 can include non-transitory computer-readable storage media, such as RAM, ROM, EEPROM (Electronically-Erasable Programmable Read-Only Memory), flash memory, optical disc storage, magnetic storage, solid state storage, or any other memory media usable to store information for later access. In examples, the memory 404 encompasses a modulated data signal (e.g., a signal that has one or more of its characteristics set or changed in such a manner as to encode information in the signal), such as a carrier wave or other transport mechanism and includes any information delivery media. By way of example, and not limitation, the memory 404 can include wired media such as a wired network or direct-wired connection, and wireless media such as acoustic, radio-frequency, infrared and other wireless media or combinations thereof.

In the illustrated example, the system 400 further includes a network adapter 406, one or more input devices 408, and one or more output devices 410. The system 400 can include other components, such as a system bus, component interfaces, a graphics system, a power source (e.g., a battery), among other components.

The network adapter 406 is a component of the computing system 400 that provides network access to network 412. The network adapter 406 can provide wired or wireless network access and can support one or more of a variety of communication technologies and protocols, such as ETHERNET, cellular, BLUETOOTH, near-field communication, and RF (Radio-frequency), among others. The network adapter 406 can include one or more antennas and associated components configured for wireless communication according to one or more wireless communication technologies and protocols.

The one or more input devices 408 are devices over which the computing system 400 receives input from a user. The one or more input devices 408 can include physically-actuatable user-interface elements (e.g., buttons, switches, or dials), touch screens, keyboards, mice, pens, and voice input devices, among others input devices.

The one or more output devices 410 are devices by which the computing system 400 is able to provide output to a user. The output devices 410 can include displays, speakers, and printers, among other output devices.

Any embodiment or any feature disclosed herein can be combined with any one or more other embodiments and/or other features disclosed herein, unless explicitly indicated otherwise. Any embodiment or any feature disclosed herein can be explicitly excluded from use with any one or more other embodiments and/or other features disclosed herein, unless explicitly indicated otherwise. It is noted that any method detailed herein also corresponds to a disclosure of a device and/or system configured to execute one or more or all of the method actions associated with the device and/or system as detailed herein. It is further noted that any disclosure of a device and/or system detailed herein corresponds to a method of making and/or using that device and/or system, including a method of using that device according to the functionality detailed herein.

The foregoing description of the exemplary embodiments of the present invention has been presented for the purpose of illustration. The foregoing description is not intended to be exhaustive or to limit the present invention to the examples disclosed herein. In some instances, features of the present invention can be employed without a corresponding use of other features as set forth. Many modifications, substitutions, and variations are possible in light of the above teachings, without departing from the scope of the present invention.

1. A system comprising:
an ultrasound device configured to generate sound waves for use in generating data indicating a measurement of a thickness of a skin flap of a recipient.

2. The system of claim 1 further comprising:
one or more processor circuits configured to generate a recommendation based on the measurement of the thickness of the skin flap regarding an external device to be placed on the recipient for coupling to an implantable device under the skin flap.

3. The system of claim 2, wherein the one or more processor circuits are further configured to generate the recommendation based on the external device having a sufficient magnetic field strength to be retained on the recipient by the implantable device.

4. (canceled)

5. The system of claim 1, wherein the ultrasound device is further configured to generate the sound waves for use in generating data indicating multiple measurements of the thickness of the skin flap, and wherein the system determines changes in the measurements over time.

6. The system of claim 1, wherein the ultrasound device is further configured to generate the sound waves for use in generating the data indicating the measurement of the thickness of the skin flap of the recipient over a potential location of an implantable component of an implant system.

7. The system of claim 1 further comprising:
a tool that determines a location on the recipient to locate the ultrasound device during the measurement of the thickness of the skin flap, wherein the tool is configured to align the ultrasound device with an implantable device implanted under the skin flap, and wherein the tool is configured to determine the location on the recipient to locate the ultrasound device based on an expected location of an external device of an implant system during normal use of the implant system.

8. (canceled)

9. (canceled)

10. (canceled)

11. The system of claim 1, wherein the ultrasound device is configured to generate the sound waves for use in generating the data indicating the measurement of the thickness of the skin flap of the recipient over an implantable component of an implant system implanted in the recipient.

12. The system of claim 1 further comprising:
a sensor configured to determine an orientation of the ultrasound device or a pressure with which the ultrasound device applies to the recipient.

13. (canceled)

14. (canceled)

15. (canceled)

16. The system of claim 1 further comprising:
one or more magnets configured to be aligned with an implantable device underneath the skin flap, wherein the one or more magnets are incorporated into at least one of the ultrasound device, an external device of an implant system that comprises the implantable device, or a tool separate from the ultrasound device.

17. (canceled)

18. A method comprising:
determining a location of a measurement of a thickness of a skin flap of a recipient based on a current or expected location of an implantable device underneath the skin flap; and
generating the measurement of the thickness of the skin flap using an ultrasound device.

19. The method of claim 18 further comprising:
determining suitability of one or more configurations of an external component of an implant system for the recipient at the current or expected location of the implantable device using the measurement of the thickness of the skin flap, wherein the implant system further comprises the implantable device.

20. The method of claim 19, wherein determining the suitability of the one or more configurations of the external component for the recipient at the current or expected location of the implantable device comprises:
determining suitability of one or more combinations of the external component and the implantable device for the recipient at the current or expected location of the implantable device based on pre-determined criteria that comprise a defined skin flap thickness range for which the one or more combinations are suitable.

21. (canceled)

22. The method of claim 18 further comprising:
determining suitability of an external component of an implant system for the recipient at the current or expected location of the implantable device using the measurement of the thickness of the skin flap, wherein the implant system further comprises the implantable device.

23. The method of claim 22, wherein determining the suitability of the external component for the recipient at the current or expected location of the implantable device further comprises:
determining the suitability of the external component for the recipient at the current or expected location of the implantable device based on pre-determined criteria that comprise at least one of a defined skin flap thickness range for which a pairing of the external component and the implantable device is suitable or expected daily activities of the recipient.

24. The method of claim 18, wherein determining the location of the measurement of the thickness of the skin flap comprises:
detecting presence of a radio-frequency coil or an antenna in the implantable device underneath the skin flap;
receiving a signal from the radio-frequency coil or the antenna; and
using properties of the signal to determine alignment with the implantable device.

25. (canceled)

26. A non-transitory computer-readable storage medium comprising instructions stored thereon that, when executed by one or more processors, cause the one or more processors to:
generate a measurement of a thickness of a skin flap of a recipient based on sonic waves applied to the skin flap; and
generate an indication of whether one or more configurations of an external device are able to be retained on the skin flap based on the measurement of the thickness of the skin flap.

27. The non-transitory computer-readable storage medium of claim 26, wherein the instructions further cause the one or more processors to:
determine if the one or more configurations of the external device have a sufficient magnetic field to be retained on the skin flap by a magnet implanted under the skin flap based on the measurement of the thickness of the skin flap.

28. The non-transitory computer-readable storage medium of claim 26, wherein the instructions further cause the one or more processors to:
analyze an ultrasound image to determine a location of one or more landmarks on an implantable device under the skin flap relative to an ultrasound device; and
generate the measurement of the thickness of the skin flap over the implantable device using the ultrasound device.

29. (canceled)

30. The non-transitory computer-readable storage medium of claim 26, wherein the instructions further cause the one or more processors to:
analyze an ultrasound image to determine a location of one or more anatomical landmarks of the recipient relative to an ultrasound device; and
generate the measurement of the thickness of the skin flap using the ultrasound device.

31. The non-transitory computer-readable storage medium of claim 26, wherein the instructions further cause the one or more processors to:
generate feedback to a user that indicates a direction or an angle to move an ultrasound device that generates the sonic waves for placement of the ultrasound device during the measurement.
